(19) United States
(12) Patent Application Publication
(10) Pub. No.: US 2024/0335245 A1
(43) Pub. Date: Oct. 10, 2024

(54) TECHNIQUES FOR ADJUSTING A FIELD OF VIEW OF AN IMAGING DEVICE BASED ON HEAD MOTION OF AN OPERATOR

(71) Applicant: INTUITIVE SURGICAL OPERATIONS, INC., Sunnyvale, CA (US)

(72) Inventors: Daniel G. MILLER, Santa Cruz, CA (US); Ian E. MCDOWALL, San Francisco, CA (US); John Ryan STEGER, Los Gatos, CA (US); Natalie BURKHARD, San Carlos, CA (US)

(21) Appl. No.: 18/294,116

(22) PCT Filed: Aug. 2, 2022
(86) PCT No.: PCT/US2022/039199

§ 371 Date: Jan. 31, 2024

Related U.S. Application Data
(60) Provisional application No. 63/228,921, filed on Aug. 3, 2021.

(51) Int. Cl. A61B 34/37; A61B 17/00; A61B 90/00

(52) U.S. Cl. CPC A61B 34/37; A61B 90/361; A61B 2017/00216; A61B 2090/368

(57) ABSTRACT

Techniques are disclosed for imaging device control in a viewing system that includes a repositionable structure configured to support an imaging device, and a control unit communicably coupled to the repositionable structure. The control unit is configured to receive head motion signals indicative of a head motion of a head of an operator relative to a reference, and in response to determining that the head motion signals indicate that the head motion does not exceed a threshold amount in a direction, cause a field of view of the imaging device to be adjusted in accordance with a commanded motion by commanding movement of at least one of the repositionable structure or the imaging device, where the commanded motion is determined based on the head motion.

### RELATED APPLICATIONS

This application claims the benefit to U.S. Provisional Application No. 63/228,921, filed Aug. 3, 2021, and entitled “Techniques for Adjusting a Field of View of an Imaging Device based on Head Motion of an Operator,” which is incorporated by reference herein.

### TECHNICAL FIELD

The present disclosure relates generally to electronic devices and more particularly to techniques for adjusting a field of view of an imaging device based on head motion of an operator.

### BACKGROUND

Computer-assisted electronic devices are being used more and more often. This is especially true in industrial, entertainment, educational, and other settings. As a medical example, the hospitals of today have large arrays of electronic devices being found in operating rooms, interventional suites, intensive care wards, emergency rooms, and/or the like. Many of these electronic devices may be capable of autonomous or semi-autonomous motion. It is also common for personnel to control the motion and/or operation of electronic devices using one or more input devices located at a user control system. As a specific example, minimally invasive, robotic telesurgical systems permit surgeons to operate on patients from bedside or remote locations. Telesurgery refers generally to surgery performed using surgical systems where the surgeon uses some form of remote control, such as a servomechanism, to manipulate surgical instrument movements rather than directly holding and moving the instruments by hand.

When an electronic device is used to perform a task at a worksite, one or more imaging devices (e.g., an endoscope) can capture images of the worksite that provide visual feedback to an operator who is monitoring and/or performing the task. The imaging device(s) may be controllable to update a view of the worksite that is provided, via a display unit, to the operator.

The display unit can be a monoscopic, stereoscopic, or three-dimensional (3D) display unit having one or more view screens. For example, the display unit could be a lenticular display that includes a pattern of cylindrical lenses in front of a liquid crystal display (LCD). To view the display unit, an operator positions his or her head so that the operator can see images on one or more view screens of the display unit. However, when the operator moves his or her head relative to the one or more view screens, a displayed view may not be changed and may even appear, from the perspective of the operator, to move in a direction that is opposite to a direction of the head motion. These effects can worsen the user experience, such as by being different to what is expected by, or familiar to, the operator, thereby causing disorientation, nausea, or visual discomfort to the operator. In addition, conventional monoscopic, stereoscopic, and 3D display units do not typically permit an operator to perceive motion parallax, or to look around an object being displayed, by moving his or her head.

Accordingly, improved techniques for adjusting the views displayed on display units of viewing systems are desirable.

### SUMMARY

Consistent with some embodiments, a computer-assisted device includes a repositionable structure configured to support an imaging device; and a control unit communicably coupled to the repositionable structure, where the control unit is configured to: receive head motion signals indicative of a head motion of a head of an operator relative to a reference, and in response to determining that the head motion signals indicate that the head motion does not exceed a threshold amount in a direction, cause a field of view of the imaging device to be adjusted in accordance with a commanded motion by commanding movement of at least one of the repositionable structure or the imaging device, wherein the commanded motion is determined based on the head motion.

Consistent with other embodiments, a method includes receiving head motion signals indicative of a head motion of a head of an operator relative to a reference; and in response to determining that the head motion signals indicate that the head motion does not exceed a threshold amount in a direction, causing a field of view of the imaging device to be adjusted in accordance with a commanded motion by commanding movement of at least one of the repositionable structure or the imaging device, where the commanded motion is determined based on the head motion.

Other embodiments include, without limitation, one or more non-transitory machine-readable media including a plurality of machine-readable instructions which when executed by one or more processors are adapted to cause the one or more processors to perform any of the methods disclosed herein.

The foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1 is a simplified diagram including an example of a computer-assisted device, according to various embodiments.

FIG. 2 illustrates an approach for detecting head motion of an operator and adjusting a field of view (FOV) of an imaging device in response to the head motion, according to various embodiments.

FIG. 3 illustrates an approach for changing an orientation of a FOV of an imaging device during adjustment to the FOV of the imaging device, according to various embodiments.

FIG. 4 illustrates an approach for detecting head motion of an operator and adjusting a FOV of an imaging device in response to the head motion, according to other various embodiments.

FIG. 5 illustrates a simplified diagram of a method for adjusting a FOV of an imaging device based on head motion of an operator, according to various embodiments.

FIG. 6 illustrates in greater detail one process of the method of FIG. 5, according to various embodiments.

### DETAILED DESCRIPTION

This description and the accompanying drawings that illustrate inventive aspects, embodiments, or modules should not be taken as limiting—the claims define the protected invention. Various mechanical, compositional, structural, electrical, and operational changes may be made without departing from the spirit and scope of this description and the claims. In some instances, well-known circuits, structures, or techniques have not been shown or described in detail in order not to obscure the invention. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, this description's terminology is not intended to limit the invention. For example, spatially relative terms-such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, or module may, whenever practical, be included in other embodiments, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, or application may be incorporated into other embodiments, or aspects unless specifically described otherwise, unless the one or more elements would make an embodiment or embodiment non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted devices and elements in terms of their state in three-dimensional space. As used herein, the term “position” refers to the location of an element or a portion of an element in a three-dimensional space (e.g., three degrees of translational freedom along Cartesian x-, y-, and z-coordinates). As used herein, the term “orientation” refers to the rotational placement of an element or a portion of an element (three degrees of rotational freedom—e.g., roll, pitch, and yaw). As used herein, the term “shape” refers to a set positions or orientations measured along an element. As used herein, and for a device with repositionable arms, the term “proximal” refers to a direction toward the base of the computer-assisted device along its kinematic chain and “distal” refers to a direction away from the base along the kinematic chain.

Aspects of this disclosure are described in reference to computer-assisted systems and devices, which may include systems and devices that are teleoperated, remote-controlled, autonomous, semiautonomous, robotic, and/or the like. Further, aspects of this disclosure are described in terms of an embodiment using a medical system including a teleoperative medical device, such as the da Vinci® Surgical System commercialized by Intuitive Surgical, Inc. of Sunnyvale, California. Knowledgeable persons will understand, however, that inventive aspects disclosed herein may be embodied and implemented in various ways, including robotic and, if applicable, non-robotic embodiments. Embodiments described for da Vinci® Surgical Systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

### System Overview

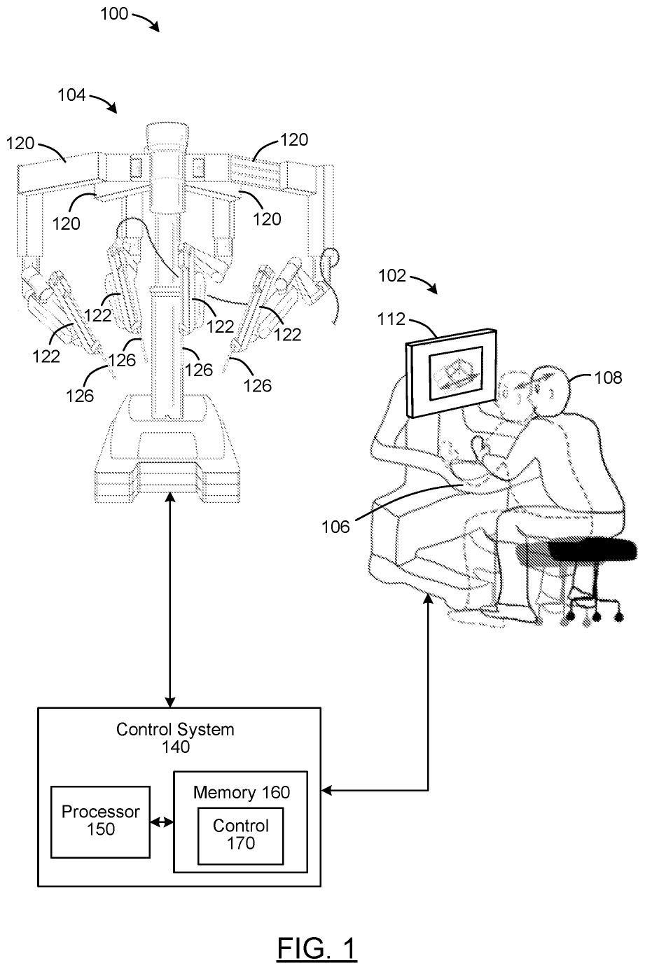

FIG. 1 is a simplified diagram of an example computer-assisted device, according to various embodiments. In some examples, the teleoperated system 100 can be a teleoperated medical system such as a surgical system. As shown, the teleoperated system 100 includes a follower device 104. The follower device 104 is controlled by one or more leader input devices, described in greater detail below. Systems that include a leader device and a follower device are also sometimes referred to as master-slave systems. Also shown in FIG. 1 is an input system that includes a workstation 102 (e.g., a console). In various embodiments, the input system can be in any appropriate form and may or may not include a workstation.

In this example, the workstation 102 includes one or more leader input devices 106 which are contacted and manipulated by an operator 108. For example, the workstation 102 can comprise one or more leader input devices 106 for use by the hands of the operator 108. The leader input devices 106 in this example are supported by the workstation 102 and can be mechanically grounded. An ergonomic support (e.g., forearm rest) can also be provided in some embodiments, on which the operator 108 can rest his or her forearms. In some examples, the operator 108 can perform tasks at a worksite near the follower device 104 during a procedure by commanding the follower device 104 using the leader input devices 106.

A display unit 112 is also included in the workstation 102. The display unit 112 displays images for viewing by the operator 108. In some embodiments, the display unit can be a monoscopic, stereoscopic, or three-dimensional (3D) display unit having one or more view screens. For example, the display unit 112 could be a lenticular display that includes a pattern of cylindrical lenses in front of a liquid crystal display (LCD) and that displays 3D holographic images. As another example, the display unit 112 could be a two-dimensional (2D) display, such as an LCD. Although described herein primarily with respect to the display unit 112 that is part of a grounded mechanical structure (e.g., the workstation 102), in other embodiments, the display unit can be any technically feasible display device or devices. For example, the display unit could be a handheld device, such as a tablet device or mobile phone. As another example, the display unit could be a head-mounted device (e.g., glasses, goggles, helmets).

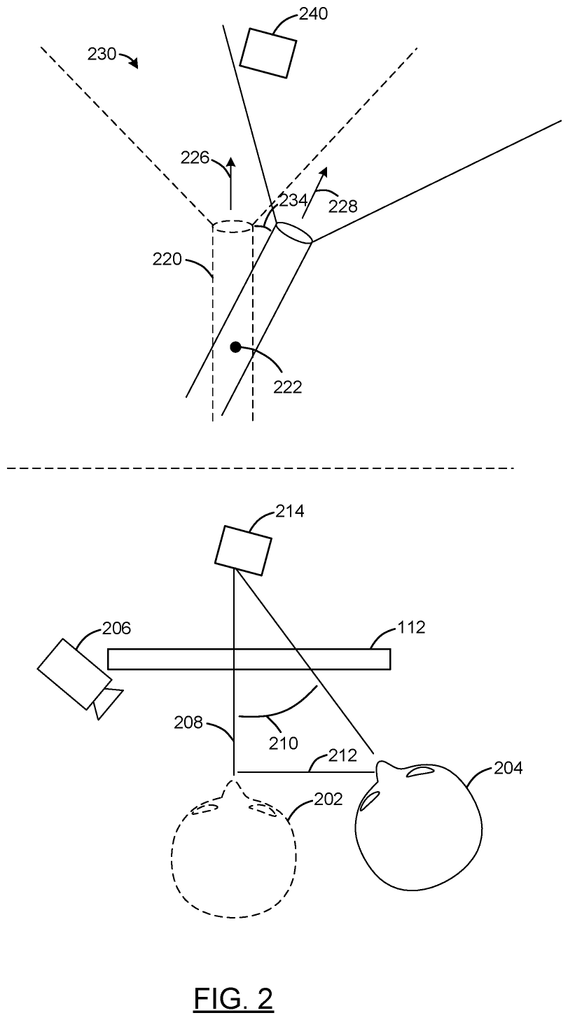

In the example of the teleoperated system 100, images displayed via the display unit 112 can depict a worksite at which the operator 108 is performing various tasks by manipulating the leader input devices 106. In some embodiments, the display unit 112 can optionally be movable in various degrees of freedom to accommodate the viewing position of the operator 108 and/or to provide control functions as another leader input device. In some examples, the images that are displayed by the display unit 112 are received by the workstation 102 from one or more imaging devices arranged in or around the worksite. In other examples, the displayed images can be generated by the display unit 112 (or by a connected other device or system), such as virtual representations of tools, or of the worksite, that are rendered from the perspective of any number of virtual imaging devices. In some embodiments, head motion of an operator (e.g., the operator 108) is detected via one or more sensors and converted into commands to cause movement of an imaging device, or to otherwise cause updating of the view in images presented to the operator (such as by graphical rendering via a virtual imaging device) via display unit 112, as described in greater detail below in conjunction with FIGS. 2-5.

When using the workstation 102, the operator 108 can sit in a chair or other support in front of the workstation 102, position his or her eyes in front of the display unit 112, manipulate the leader input devices 106, and rest his or her forearms on an ergonomic support as desired. In some embodiments, the operator 108 can stand at the workstation or assume other poses, and the display unit 112 and leader input devices 106 can be adjusted in position (height, depth, etc.) to accommodate the operator 108.

In some embodiments, one or more leader input devices can be ungrounded (ungrounded leader input devices being not kinematically grounded, such as leader input devices held by the hands of the operator 108 without additional physical support). Such ungrounded leader input devices can be used in conjunction with the display unit 112. In some embodiments, the operator 108 can use a display unit 112 positioned near the worksite, such that the operator 108 can manually operate instruments at the worksite, such as a laparoscopic instrument in a surgical example, while viewing images displayed by the display unit 112.

The teleoperated system 100 also includes the follower device 104, which can be commanded by the workstation 102. In a medical example, the follower device 104 can be located near an operating table (e.g., a table, bed, or other support) on which a patient can be positioned. In such cases, the worksite can be provided on the operating table, e.g., on or in a patient, simulated patient or model, etc. (not shown). The teleoperated follower device 104 shown includes a plurality of manipulator arms 120, each configured to couple to an instrument assembly 122. An instrument assembly 122 can include, for example, an instrument 126 and an instrument carriage configured to hold a respective instrument 126.

In various embodiments, one or more of the instruments 126 can include an imaging device for capturing images. For example, one or more of the instruments 126 could be an endoscope assembly that includes one or more optical cameras, hyperspectral cameras, ultrasonic sensors, etc. which can provide captured images of a portion of the worksite to be displayed via the display unit 112.

In some embodiments, the follower manipulator arms 120 and/or instrument assemblies 122 can be controlled to move and articulate the instruments 126 in response to manipulation of leader input devices 106 by the operator 108, so that the operator 108 can perform tasks at the worksite. The manipulator arms 120 and instrument assemblies 122 are examples of repositionable structures on which instruments and/or imaging devices can be mounted. For a surgical example, the operator can direct the follower manipulator arms 120 to move one or more of the instruments 126 to perform surgical procedures at internal surgical sites through minimally invasive apertures or natural orifices.

As shown, a control system 140 is provided external to the workstation 102 and communicates with the workstation 102 and the follower device 104. In other embodiments, the control system 140 can be provided in the workstation 102 or in the follower device 104. As the operator 108 moves leader input device(s) 106, sensed spatial information including sensed position and/or orientation information is provided to the control system 140 based on the movement of the leader input devices 106. The control system 140 can determine or provide control signals to the follower device 104 to control the movement of the manipulator arms 120, instrument assemblies 122, and/or instruments 126 based on the received information and operator input. In one embodiment, the control system 140 supports one or more wired communication protocols, (e.g., Ethernet, USB, and/or the like) and/or one or more wireless communication protocols (e.g., Bluetooth, IrDA, HomeRF, IEEE 802.11, DECT, Wireless Telemetry, and/or the like).

The control system 140 can be implemented on one or more computing systems. One or more computing systems can be used to control the follower device 104. In addition, one or more computing systems can be used to control components of the workstation 102, such as movement of the display unit 112.

As shown, the control system 140 includes a processor 150 and a memory 160 storing a control module 170. In some embodiments, the control system 140 can include one or more processors, non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, etc.), a communication interface (e.g., Bluetooth interface, infrared interface, network interface, optical interface, etc.), and numerous other elements and functionalities. In addition, functionality of the control module 170 can be implemented in any technically feasible software and/or hardware.

Each of the one or more processors of the control system 140 can be an integrated circuit for processing instructions. For example, the one or more processors can be one or more cores or micro-cores of a processor, a central processing unit (CPU), a microprocessor, a field-programmable gate array (FPGA), an application-specific integrated circuit (ASIC), a digital signal processor (DSP), a graphics processing unit (GPU), a tensor processing unit (TPU), and/or the like. The control system 140 can also include one or more input devices, such as a touchscreen, keyboard, mouse, microphone, touchpad, electronic pen, or any other type of input device.

A communication interface of the control system 140 can include an integrated circuit for connecting the computing system to a network (not shown) (e.g., a local area network (LAN), a wide area network (WAN) such as the Internet, mobile network, or any other type of network) and/or to another device, such as another computing system.

Further, the control system 140 can include one or more output devices, such as a display device (e.g., a liquid crystal display (LCD), a plasma display, touchscreen, organic LED display (OLED), projector, or other display device), a printer, a speaker, external storage, or any other output device. One or more of the output devices can be the same or different from the input device(s). Many different types of computing systems exist, and the aforementioned input and output device(s) can take other forms.

Software instructions in the form of computer readable program code to perform embodiments of the disclosure can be stored, in whole or in part, temporarily or permanently, on a non-transitory computer readable medium such as a CD, DVD, storage device, a diskette, a tape, flash memory, physical memory, or any other computer readable storage medium. Specifically, the software instructions can correspond to computer readable program code that, when executed by a processor(s), is configured to perform some embodiments of the invention.

Continuing with FIG. 1, the control system 140 can be connected to or be a part of a network. The network can include multiple nodes. The control system 140 can be implemented on one node or on a group of nodes. By way of example, the control system 140 can be implemented on a node of a distributed system that is connected to other nodes. By way of another example, the control system 140 can be implemented on a distributed computing system having multiple nodes, where different functions and/or components of the control system 140 can be located on a different node within the distributed computing system. Further, one or more elements of the aforementioned control system 140 can be located at a remote location and connected to the other elements over a network.

Some embodiments can include one or more components of a teleoperated medical system such as a da Vinci® Surgical System, commercialized by Intuitive Surgical, Inc. of Sunnyvale, California, U.S.A. Embodiments on da Vinci® Surgical Systems are merely examples and are not to be considered as limiting the scope of the features disclosed herein. For example, different types of teleoperated systems having follower devices at worksites, as well as non-teleoperated systems, can make use of features described herein.

### Adjusting the Field of View of an Imaging Device Based on Operator Head Motion

As described, in some embodiments, a workstation can include one or more sensors that sense head motion of an operator, and the head motion can be converted to commands that cause a field of view (FOV) of an imaging device to be adjusted, or cause in some other manner the updating of the view in images presented to the operator (e.g., images rendered using a virtual imaging device) via a display unit.

FIG. 2 illustrates an approach for detecting head motion of an operator and adjusting the FOV of an imaging device in response to the head motion, according to various embodiments. As shown, a head motion of an operator (e.g., the operator 108) from a reference position 202 to a new position 204 is tracked via a sensor 206 and converted to a corresponding adjustment to a FOV 230 of an imaging device 220 from a reference FOV pose (i.e., position and/or orientation) to a new FOV pose, represented as vectors whose directions indicate centers of the reference FOV pose 226 and the new FOV pose 228. As used herein, an adjustment to the FOV of an imaging device can include translational motion (i.e., a change in position), rotational motion (i.e., a change in orientation), or a combination thereof. In some examples, the imaging device 220 includes one or more devices (not shown) for capturing images, such as one or more cameras that detect in infrared, visible, or ultraviolet spectrum, ultrasonic sensors, etc. that comprise part of a tool. For example, the imaging device 220 could be an endoscope that includes an optical camera. In other examples, the imaging device 220 can be a virtual imaging device that is used to render 3D virtual, augmented, or mixed reality environments. As shown in the example of FIG. 2, adjusting the FOV 230 of the imaging device 220 to the new FOV pose 228 permits images to be captured (or rendered) from a vantage point to the right of a vantage point associated with the reference FOV pose 226. As a result, the imaging device 220 can capture images that are closer to what is expected by, or familiar to, the operator whose head position has moved rightward from the reference position 202 to the new position 204.

The sensor 206 is representative of any technically feasible sensor, or sensors, configured to sense the position and/or motion of the head of an operator. In some examples, the sensor 206 can include a time-of-flight sensor, such as a Light Detection and Ranging (LiDAR) sensor, a computer-vision based sensor, an accelerometer or inertial sensor coupled directly or indirectly to the head, a camera, an emitter-receiver system with the emitter or received coupled directly or indirectly to the head, or a combination thereof. The position and/or motion of the head of an operator can be tracked in any technically feasible manner using the sensor 206. In some examples, signals received from the sensor 206 are used to detect the head of the operator as a blob using well-known techniques, and a position associated with the blob can be tracked over time to determine the head motion. In other examples, particular features on the head of an operator, such as the eyes of the operator, can be tracked. In addition, the head motion of the operator can be tracked in one dimension (e.g., left and right motions), two dimensions (e.g., right/left and up/down), or three dimensions (e.g., right/left, up/down and forward/backward), in some embodiments. In some embodiments, the head motion can be derived using techniques that aggregate, filter, or average sensor signals over space (e.g., from multiple sensing elements) or time.

In some embodiments, the control module 170 determines, based on signals that are received from the sensor 206, left-right and up-down displacements (i.e., displacements that are not toward or away from the display unit 112 in a forward-backward direction) of the head of the operator relative to the reference position 202. For each of the left-right and up-down displacements, an angle associated with the displacement can be determined based on an arctangent of the displacement divided by a distance from the head of the operator to a representation of an object 214 displayed via the display unit 112. As shown in the example of FIG. 2, an angle 210 associated with a rightward head motion from the reference position 202 to the new position 204 can be calculated as the arctangent of a displacement 212 between the positions 202 and 204 divided by a distance 208 from the head of the operator at the reference position 202 to the representation of the object 214. Similar calculations can be performed to determine angles associated with left, up, and down head displacements (not shown). In some embodiments, the distance from the head of the operator to the representation of the object 214 can be determined by (1) measuring, via the sensor 206, a distance of the head of the operator from the display unit 112; and (2) adding the measured distance to a known distance that the representation of the object 214 is behind the display unit 112 (i.e., in a direction away from the operator) in one or more images that are displayed. The distance of the head of the operator to the representation of the object 214 can change if the operator moves his or her head closer to or farther away from the display unit 112 in a forward-backward direction.

The control module 170 further determines whether each angle associated with the left-right and up-down displacements is greater than a minimum threshold angle. In some examples, the minimum threshold angle can be 0.25-0.5 degrees. When the angle associated with the left-right or the up-down displacement is not greater than the minimum threshold angle, then the displacement can be ignored so that the imaging device 220 is not being constantly moved in response to relatively small head motions of the operator.

When the angle associated with a left-right or up-down displacement is greater than the minimum threshold angle, then the control module 170 further determines whether the angle is less than a maximum threshold angle. Head movements beyond the maximum threshold angle are not followed by FOV 230 of the imaging device 220, because the FOV 230 of the imaging device 220 is not intended to follow all head movements, and the imaging device 220 can also be physically unable to follow relatively large head movements. In some embodiments, the FOV 230 of the imaging device 220 is rotated in the yaw and pitch directions to follow the angles of head motions in the left-right and up-down directions, respectively, within a range of angles up to the maximum threshold angle of motion for each direction. In some examples, the maximum threshold angle can be 5-7 degrees of head movement by the operator. In addition, in some embodiments, the FOV 230 of the imaging device 220 can remain unchanged, or prior adjustments can be reversed, if the head motion exceeds the maximum threshold angle (or another threshold angle) within a certain period of time or if a gaze of the operator is detected to no longer be directed towards the display unit 112, such as if the operator turned his or her head to speak to someone nearby.

When the angle associated with the left-right and/or up-down displacements is less than the maximum threshold angle, then the control module 170 determines a corresponding yaw and/or pitch angle for adjusting the FOV 230 of the imaging device 220 relative to the reference FOV pose 226 that allows the FOV 230 of the imaging device 220 to follow the head motion of the operator. In some embodiments, the angles associated with the left-right and up-down displacements are negatively scaled to determine corresponding angles by which to yaw or pitch the FOV 230 of the imaging device 220, respectively. In some examples, the scaling can be one-to-one, non-linear when an angle is near zero to avoid issues at relatively small angles, and/or dependent on optical parameters associated with the imaging device 220. The optical parameters associated with the imaging device 220 can include a focal distance of a sensor (e.g., an optical camera, hyperspectral camera, ultrasonic sensor, etc.) included in the imaging device 220, a type of the sensor (e.g., whether an optical camera is a wide-angle camera), etc. For example, if the imaging device 220 includes a zoomed-in camera that is associated with a relatively long focal length, then a scaling factor could be selected that adjusts the FOV 230 of the imaging device 220 relatively little in response to head motions of the operator. In some embodiments, a different scaling factor can be applied to left-right head motions than to up-down head motions of the operator.

As shown, the angle 210 associated with the head displacement 212 from the reference position 202 to the new position 204 is negatively scaled to obtain the angle 234 by which to adjust the FOV 230 of the imaging device 220 relative to the reference FOV pose 226. As a result of the negative scaling, the FOV 230 of the imaging device 220 is rotated in a clockwise yaw direction for a rightward movement of the head of the operator, which corresponds to a counterclockwise rotation in terms of the angle 210, and vice versa for a leftward movement of the head of the operator. Similarly, the FOV 230 of the imaging device 220 can be rotated in a clockwise pitch direction for an upward movement of the head of the operator, which corresponds to a counterclockwise rotation, and vice versa for a downward movement of the head of the operator. As described, in the example of FIG. 2, when the head of the operator moves in the rightward direction relative to the reference position 202, then the FOV 230 of the imaging device 220 is also moved to capture images from a vantage point to the right of a vantage point associated with the reference FOV pose 226. As a result, the imaging device 220 can capture images that are closer to what is expected by, or familiar to, the operator, thereby reducing or eliminating nausea and visual discomfort to the operator. In addition, the captured images permit the operator to perceive motion parallax and occlusions in the images, as well as to look around an object 240 that is captured and displayed via the display unit 112 as the representation of the object 214.

After angles of motion in the yaw and pitch directions are determined, the imaging device 220 is moved to achieve those angles based on inverse kinematics of the imaging device 220 and/or a repositionable structure to which the imaging device 220 is mounted. In some examples, the control module 170 can use inverse kinematics to determine how joints of the imaging device 220 and/or the repositionable structure to which the imaging device 220 is mounted can be actuated so that the imaging device 220 is adjusted to a position associated with the FOV pose 228 that is at the angle 234 relative to the reference FOV pose 226. The control module 170 can then issue commands to controllers for the joints of the imaging device 220 and/or the repositionable structure to cause movement of the imaging device 220.

In the example of FIG. 2, the imaging device 220 is an endoscope including one or more optical cameras (not shown). The cameras provide captured images of a portion of a worksite that are displayed to the operator via the display unit 112. In other embodiments, the imaging device can be a virtual camera that is used to render at least a portion of a 3D virtual environment. As shown, the imaging device 220 is constrained to pivot about a pivot point 222 and to roll about an axis that lies along a center line of a shaft of the imaging device 220. For example, the pivot point 222 could be a point on a body wall at which the endoscope is inserted into a patient or an access port where the imaging device 220 is inserted into a workspace. As described, the imaging device 220 is rotated about the pivot point 222 such that the FOV 230 of the imaging device rotates away from the reference FOV pose 226 by the angle 234 to the new FOV pose 228 in response to head movement of the operator from the reference position 202 to the new position 204. As shown, the reference FOV pose 226 is different from the new FOV pose 228 provided by the imaging device 220 after the imaging device 220 is moved.

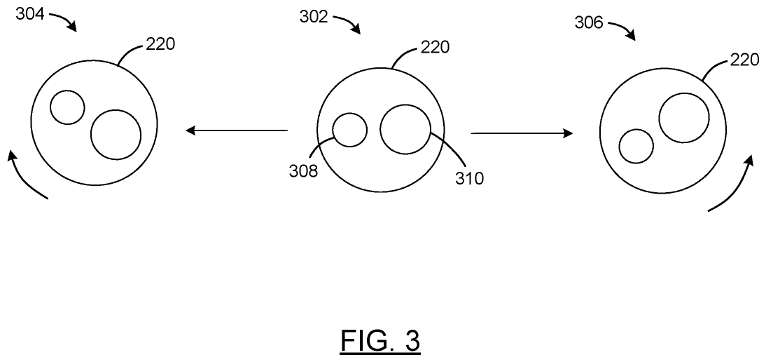

In addition to rotating the imaging device 220 about the pivot point 222, in some embodiments, the control module 170 determines a change in orientation of the imaging device 220 based on the left-right displacement of the head of the operator. FIG. 3 illustrates an approach for changing an orientation of an imaging device during adjustment of the FOV of the imaging device, according to various embodiments. As shown, the imaging device 220 that includes sensor devices 308 and 310 (which can be, e.g., optical cameras, hyperspectral cameras, ultrasonic sensors, etc.) can be repositioned to adjust a FOV 230 of the imaging device 220 that is captured by the sensor devices 308 based on left-right displacements of the head of an operator. In the example of FIG. 3, when the FOV 230 of the imaging device 220 is adjusted due to a repositioning of the imaging device 220 from an original position 302 to a leftward position 304 or to a rightward position 306 based on left-right displacements of the head of the operator, the FOV 230 of the imaging device 220 is further adjusted by rolling the FOV 230 in a clockwise direction or in a counterclockwise direction, respectively, based on the left-right displacements. In some embodiments, a roll angle of the FOV 230 of the imaging device 220, which is a change in angular position relative to a reference orientation of the FOV 230 in the reference FOV pose 226, is proportional to the left-right displacement of the head of the operator. In such cases, a proportional gain of the roll can be 0.25, or based on an empirically determined gain value. By rolling the FOV 230 of the imaging device 220, images can be captured (or generated in the case of a virtual imaging device) by the imaging device 220 that are closer to what is familiar, or expected, by an operator. For example, the roll can mimic a slight rotation of the head of the operator that likely occurs along with the translation of the head of the operator from the reference position 202 to the new position 204 as the operator pivots his or her head at the neck.

Returning to FIG. 2, in some embodiments, when the angle associated with a left-right or up-down head displacement from the reference position 202 is greater than the maximum threshold angle, then the FOV 230 of the imaging device 220 is only rotated to a maximum yaw or pitch angle associated with the maximum threshold angle. As described, the maximum threshold angle can be 5-7 degrees in some embodiments. Head movements beyond the maximum threshold angle are not followed by the FOV 230 of the imaging device 220 because the FOV 230 of the imaging device 220 is not intended to follow all head movements, and the FOV 230 of the imaging device 220 can also be physically unable to follow large head movements. The maximum threshold angle can be the same, or different for left-right and up-down head motions in some embodiments. The FOV 230 of the imaging device 220 is only adjusted to follow left-right or up-down head motions of the operator up to the corresponding maximum threshold angle. When the head position of the operator in the left-right or up-down direction returns to a displacement from the reference position 202 associated with an angle that is less than the maximum threshold angle, the FOV 230 of the imaging device 220 can again be adjusted based on the head motions of the operator. In other embodiments, the FOV 230 of the imaging device 220 can be returned to the reference FOV pose 226 when an angle associated with a left-right or up-down head displacement from the reference position 202 exceeds a corresponding maximum threshold angle.

In some embodiments, the left-right and up-down reference positions (e.g., reference position 202) with respect to which head motions of the operator are determined can be reset when the maximum threshold angle, described above, is exceeded for a threshold period of time. In some examples, the threshold period of time can be a few minutes. By resetting the reference position 202 after the head motion exceeds the maximum threshold angle for the threshold period of time, later head motions of the operator can be determined relative to a current head position of the operator after the head of the operator moves from one position to another. For example, the operator could move in his or her chair to a different head position and stay in that position for more than the threshold period of time. In such a case, the reference position 202 would be reset to the current head position. In some embodiments, when resetting the reference position 202, a low-pass filter can be applied to the head motion of the operator after the maximum threshold angle is exceeded for the threshold period of time. For example, a low-pass filter could be used to gently move the reference position to the current position of the head of the operator through multiple steps over a configurable period of time, such as 10 seconds.

In some embodiments, the reference FOV pose 226 with respect to which adjustments of the FOV 230 of the imaging device 220 are determined can be reset at the end of an imaging device repositioning operation. In some examples, the operator is permitted to change the position and/or orientation of the FOV 230 of the imaging device 220 using one or more hand and/or foot input controls. When the operator is changing the position and/or orientation of the FOV 230 of the imaging device 220, the imaging device 220 is adjusted according to commands generated in response to the hand and/or foot input controls, rather than head motions sensed via the sensor 206, i.e., the hand and/or foot input controls supersede the head motions. At the end of the imaging device repositioning operation, the reference FOV pose 226 of the imaging device 220 can be reset to the current FOV of the imaging device 220.

In some embodiments, various parameters described herein, such as the minimum and maximum thresholds for the head motion, the scaling factors, the threshold period of time, etc., can be determined based on one or more of a type of the imaging device 220, a type of the display unit 112, a type of the repositionable structure, operator preference, a type of a procedure being performed at the worksite, a focal length of the imaging device 220, among other things.

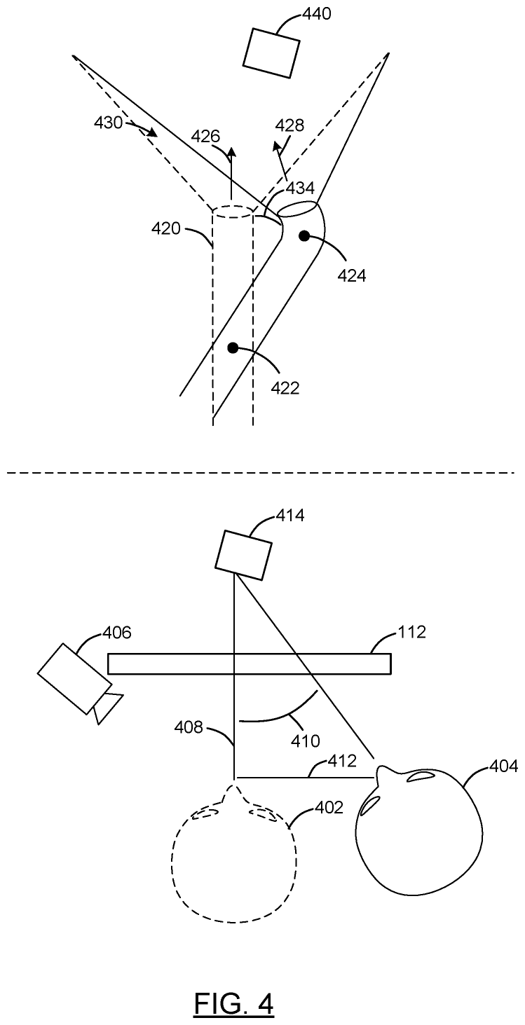

FIG. 4 illustrates an approach for detecting head motion of an operator and adjusting the FOV of an imaging device in response to the head motion, according to other various embodiments. As shown, a head motion of an operator from a reference position 402 to a new position 404 is converted to an adjustment to the FOV 430 of an imaging device 420, similar to the description above for FIG. 2. In some embodiments, angles (e.g., angle 434) by which FOV 430 of the imaging device 420 yaws and pitches are determined by negatively scaling angles (e.g., angle 410) associated with left-right and up-down displacements of the head of the operator, respectively.

As shown, the imaging device 420 is an endoscope including one or more optical cameras that are mounted at a distal end of the endoscope and provide captured images of a portion of a worksite that are displayed to an operator via the display unit 112. Similar to the imaging device 220, the imaging device 420 can pivot about a pivot point 422 and roll about an axis that lies along a center line of a shaft of the imaging device 420. Unlike the imaging device 220, the imaging device 420 includes a flexible wrist that permits a distal end of the imaging device 420 to pivot about another point 424. In other embodiments, a flexible wrist can permit an imaging device to bend in any technically feasible manner.

Illustratively, in addition to computing the angle 434 by which to rotate the imaging device 420, the control module 170 further determines an articulation of the wrist of the imaging device 420 that aligns a direction of the FOV 430 of the imaging device 420 with a direction of view of the operator to a representation of an object 414 displayed via the display unit 112. The direction of view of the operator can be specified by the same angle 410 with respect to the reference position 402. As shown, the wrist of the imaging device 420 has been articulated, based on the direction of view of the operator, to point the FOV 430 of the imaging device 420 toward the object 440 being captured by the imaging device 420. As a result, a reference FOV pose 426 provided by the imaging device before being adjusted is substantially the same as a new FOV pose 428 provided by the imaging device 420 after the imaging device 420 is moved. As shown, the reference FOV pose 426 and the new FOV pose 428 are represented as vectors whose directions indicate centers of the reference FOV pose 426 and the new FOV pose 428, respectively.

Because the direction of the FOV 430 of the imaging device 420 is aligned with the direction of view of the operator, the FOV 430 of the imaging device 420 is not rolled based on left-right head motions of the operator in some embodiments, in contrast to the FOV 230 of the imaging device 220 described above in conjunction with FIGS. 2-3. In other embodiments in which an articulated wrist of an imaging device cannot fully align the direction of the FOV of an imaging device with the direction of view of an operator (due to, e.g., range of motion limits of the wrist), the FOV of the imaging device can still be rolled based on left-right head motions of the operator.

Although described herein primarily with respect to determining an articulation of a wrist in addition to angles by which to rotate the FOV of an imaging device that includes a flexible wrist, in other embodiments, a flexible wrist can be articulated based on the head motion of an operator to adjust a FOV of an imaging device that captures images based on the head motion, as well as to align with a direction of view of the operator after the head motion. In such cases, the head motion can be directly mapped to the wrist motion that adjusts the FOV of the imaging device, without requiring the image device to be rotated about a pivot point, such as the pivot point 422.

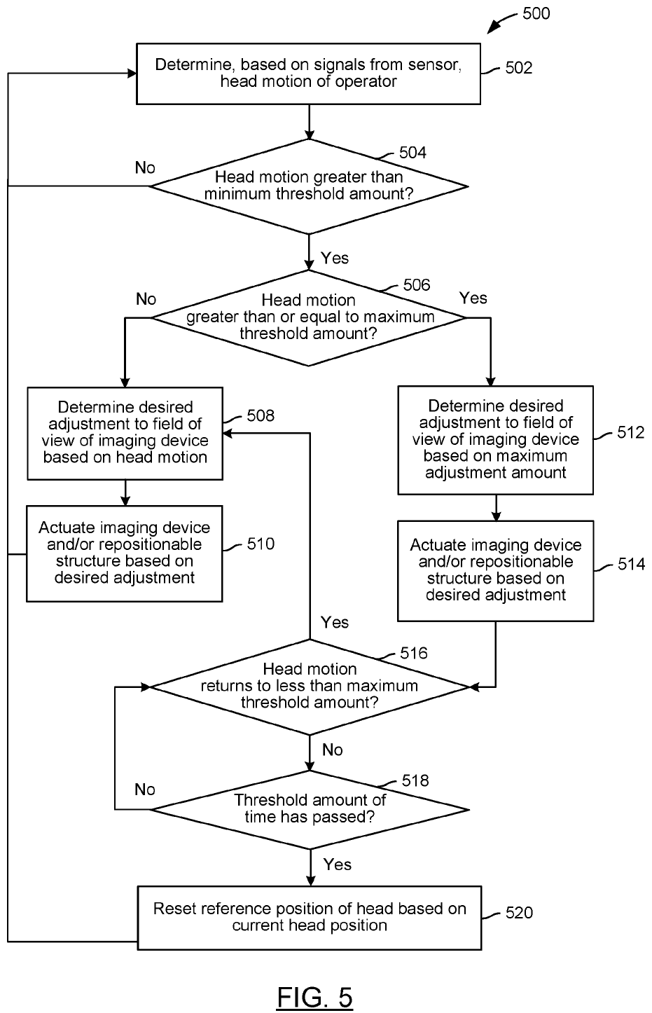

Although described herein primarily with respect to computing an angle (e.g., the angle 210 or 410) associated with a head motion and adjusting the FOV of an imaging device based on the angle, in some embodiments, an adjustment to the FOV of an imaging device can be determined in other ways based on head motion of an operator. For example, in some embodiments, a head displacement (e.g., the displacement 212 or 412) relative to a reference position of the head of an operator can be converted directly to a displacement of the FOV of an imaging device by negatively scaling the head displacement based on a ratio between the distance of the operator from an object being displayed by a display unit and the distance of an imaging device from an object being captured at a worksite, without computing an associated angle. In some embodiments, changes in the distance of the operator from the object being displayed can be included and/or omitted during the determination of how much to adjust the FOV of the imaging device. FIG. 5 illustrates a simplified diagram of a method 500 for adjusting a FOV of an imaging device based on head motion of an operator, according to various embodiments. One or more of processes 502-520 of the method 500 can be implemented, at least in part, in the form of executable code stored on non-transitory, tangible, machine readable media that when run by one or more processors (e.g., the processor 150 in control system 140) can cause the one or more processors to perform one or more of the processes 502-520. In some embodiments, the method 500 can be performed by one or more modules, such as control module 170 in the control system 140. In some embodiments, the method 500 can include additional processes, which are not shown. In some embodiments, one or more of the processes 502-520 can be performed, at least in part, by one or more of the modules of the control system 140.

A FOV of an imaging device can be adjusted based on head motion of an operator according to the method 500 in various operating modes. In some embodiments, the FOV of the imaging device can always be adjusted in response to head motions of the operator. In other embodiments, a mode in which the FOV of the imaging device is adjusted in response to head motions of the operator can be enabled or disabled based on an operating mode of a system including an imaging device, operator preference, and/or the like. In some embodiments, the FOV of the imaging device can be adjusted based on a combination of head motions of the operator and control inputs received via one or more other input modalities, such as by superimposing adjustments based on the head motions of the operator and adjustments based on the control inputs received via the one or more other input modalities. For example, the one or more other input modalities could include a hand-operated controller, such as one of the leader input devices 106 described above in conjunction with FIG. 1, and/or a foot-operated controller.

As shown, the method 500 begins at process 502, where a head motion of an operator is determined based on signals from a sensor (e.g., sensor 206 or 406). In some embodiments, the head motion can be an angle relative to a reference position that is determined as an arctangent of the displacement divided by a distance from the head of the operator to a representation of an object displayed via a display unit (e.g., display unit 112), as described above in conjunction with FIG. 2. In some embodiments, the head motion at process 502 is either a left-right or an up-down movement of the head of the operator that is used to determine corresponding left-right or up-down angles for adjusting the FOV of an imaging device, respectively. In such cases, processes 502-520 of the method 500 can be repeated to adjust the FOV of the imaging device based on head motions in the other (left-right or up-down) direction. In other embodiments, the head motion at process 502 can include both a left-right and an up-down movement of the head of the operator that is used to determine both left-right and up-down adjustments to the FOV of an imaging device. In some embodiments, an angle of motion is not computed, and a left-right and/or up-down displacement from the reference position can be directly used as the head motion.

At process 504, it is determined whether the head motion is greater than a minimum threshold amount of motion. As described, in some embodiments, the minimum threshold amount of motion can be a minimum threshold angle of 0.25-0.5 degrees, or a minimum displacement, in each of the left-right and up-down directions. In such cases, the angle or the displacement associated with the head motion in the left-right and/or up-down directions, described above in conjunction with process 502, can be compared with the corresponding minimum threshold angle.

When the head motion is not greater than the minimum threshold amount of motion, then the FOV of an imaging device (e.g., imaging device 220) is not adjusted based on the head motion, and the method 500 returns to process 502. When the head motion is greater than the minimum threshold amount of motion, then the method 500 continues to process 506, where it is determined whether the head motion is greater than or equal to a maximum threshold amount of motion. Similar to process 504, in some embodiments, the maximum threshold amount of motion can be a maximum threshold angle of 5-7 degrees, or a maximum displacement, in each of the left-right and up-down directions. In such cases, the angle or displacement associated with the head motion in the left-right and/or up-down directions, described above in conjunction with process 502, can be compared with the corresponding maximum threshold angle.

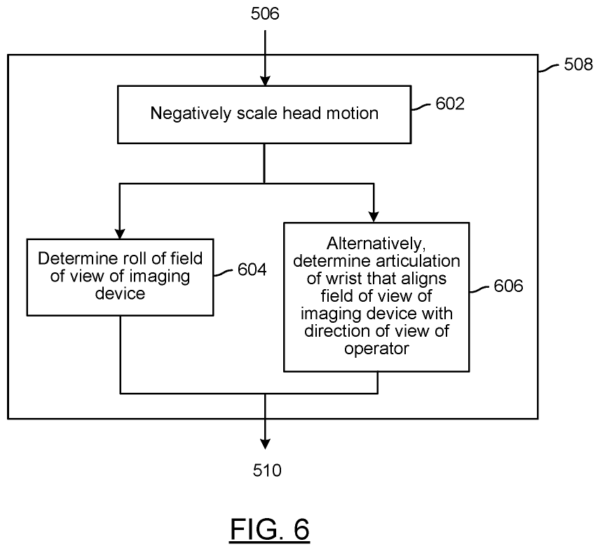

When the head motion is not greater than or equal to the threshold amount of motion, then at process 508, a desired adjustment to the FOV of the imaging device is determined based on the head motion. FIG. 6 illustrates in greater detail process 508 of the method of FIG. 5, according to various other embodiments. As shown, at process 602, the head motion is negatively scaled. In some embodiments, an angle or displacement of the head motion relative to a reference position is negatively scaled to determine a corresponding angle or displacement for adjusting the FOV of the imaging device relative to a reference FOV pose of the imaging device, as described above in conjunction with FIG. 2. In such cases, the scaling can be one-to-one, non-linear when an angle (or displacement) is near zero to avoid issues at relatively small angles (or displacements), and/or dependent on optical parameters associated with the imaging device.

At process 604, a roll of the FOV of the imaging device is determined. Process 604 can be performed in some embodiments in which the imaging device does not include a flexible wrist. In some embodiments, a left-right displacement of the head of an operator is scaled to determine a roll angle for the FOV of the imaging device relative to a reference orientation of the FOV of the imaging device, as described above in conjunction with FIG. 3. In such cases, a proportional gain of the roll relative to the left-right head displacement can be 0.25, or based on an empirically determined gain value.

Alternatively, at process 606, an articulation of a wrist that aligns the FOV of the imaging device with a direction of view of the operator is determined. Process 606 can be performed instead of process 604 in some embodiments in which the imaging device includes a flexible wrist. In other embodiments, head motion of an operator can be directly mapped to motion of the wrist of an imaging device, without requiring the FOV of the image device to be rotated about a pivot point.

Returning to FIG. 5, at process 510, the imaging device and/or a repositionable structure to which the imaging device is mounted is actuated based on the desired adjustment to the FOV of the imaging device. In some embodiments, one or more commands can be determined and issued to controllers for joints in the imaging device (e.g., joints associated with an articulated wrist) and/or the repositionable structure to cause movement of the imaging device to achieve the desired adjustment to the FOV of the imaging device, as described above in conjunction with FIG. 2.

When the head motion is determined at process 506 to be greater than or equal to the maximum threshold amount of motion, then at process 512, an adjustment to the FOV of the imaging device is determined based on a maximum adjustment amount. In some examples, the maximum adjustment amount is a maximum angle relative (or a maximum displacement in some embodiments in which an angle is not calculated) to a reference FOV pose of the imaging device that the FOV can be rotated based on the head motion. In other embodiments, the FOV of the imaging device can be returned to a reference FOV pose when the head motion is greater than the maximum threshold amount of motion.

At process 514, the imaging device and/or a repositionable structure to which the imaging device is mounted is actuated based on the desired adjustment to the FOV of the imaging device. Process 514 is similar to process 510, described above.

At process 516, when the head motion returns to less than the maximum threshold amount of motion, the method 500 continues to process 508, where a desired adjustment to the FOV of the imaging device is determined based on the head motion. However, when the head motion does not return to less than the maximum threshold amount of motion, and a threshold amount of time has passed at process 518, then the reference position of the head of the operation is reset based on a current head position at process 520.

As described in various ones of the disclosed embodiments, head motions of an operator relative to a reference position are tracked, and the FOV of an imaging device is adjusted based on the head motions, up to a threshold adjustment amount. In some embodiments, the head motions include angles that are determined based on displacements of the head of the operator in left-right and up-down directions. In such cases, the FOV of the imaging device is rotated in the yaw and pitch directions to follow the angles of the head motions in left-right and up-down directions, respectively, within a range of angles up to a maximum angle for each direction. In other embodiments, the FOV of the imaging device can be displaced based on a displacement of the head of the operator in the left-right and up-down directions within a range of displacements up to a maximum displacement for each direction. In addition, references from which head motions and adjustments to the FOV of the imaging device are determined can be reset for each direction when the head position exceeds the corresponding maximum angle or displacement for a threshold period of time and at the end of a repositioning operation of the FOV of the imaging device, respectively.

Advantageously, the disclosed techniques can provide a response to motions of the head of an operator that is closer to what is familiar to, or expected, by the operator relative to views displayed by conventional display units. For example, the disclosed techniques can be implemented to permit an operator to perceive motion parallax and to look around an object being displayed by moving his or her head. In addition, the disclosed techniques can be implemented to reduce or eliminate discomfort to the operator that can be caused when a displayed view does not change in a manner similar to that of physical objects, such as when the displayed view is not changed in response to head motion of the operator, and such as when the displayed view moves, from the perspective of the operator, in a direction that is opposite to the head motion.

Some examples of control systems, such as control system 140 can include non-transitory, tangible, machine readable media that include executable code that when run by one or more processors (e.g., processor 150) can cause the one or more processors to perform the processes of method 500 and/or the processes of FIGS. 5-6. Some common forms of machine readable media that can include the processes of method 500 and/or the processes of FIGS. 5-6 are, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and, in a manner, consistent with the scope of the embodiments disclosed herein.

1. A computer-assisted device comprising:
a repositionable structure configured to support an imaging device; and
a control unit communicably coupled to the repositionable structure;
wherein the control unit is configured to:
receive head motion signals indicative of a change in position of a head of an operator relative to a reference; and
in response to determining that the head motion signals indicate that the change in position does not exceed a threshold amount in a direction, cause an orientation of a field of view (FOV) of the imaging device to be adjusted in accordance with a commanded motion by commanding movement of at least one of the repositionable structure or the imaging device, wherein the commanded motion is determined based on the change in position.

2. The computer-assisted device of claim 1, wherein the control unit is further configured to determine the commanded motion by:
determining a first angle associated with the change in position in a left-right direction relative to the reference;
determining, based on the first angle, a second angle by which to adjust the FOV of the imaging device in a yaw direction relative to a FOV reference pose; and
determining the commanded motion based on the second angle.

3. The computer-assisted device of claim 2, wherein determining the second angle comprises negatively scaling the first angle according to a first scaling factor.

4. (canceled)

5. The computer-assisted device of claim 2, wherein the control unit is further configured to determine the commanded motion by:
determining a third angle associated with the change in position in an up-down direction relative to the reference;
determining, based on the third angle, a fourth angle by which to adjust the FOV of the imaging device in a pitch direction relative to the FOV reference pose; and
determining the commanded motion further based on the fourth angle.

6-7. (canceled)

8. The computer-assisted device of claim 2, wherein the control unit is further configured to determine the commanded motion by:
determining a fifth angle by which to roll the FOV of the imaging device based on the change in position in the left-right direction relative to the reference; and
determining the commanded motion further based on the fifth angle.

9. (canceled)

10. The computer-assisted device of claim 2, wherein the imaging device comprises a shaft and a joint disposed at a distal portion of the shaft, and wherein the control unit is further configured to determine the commanded motion by:
determining an articulation of the joint that would align a direction of the FOV of the imaging device with a direction of view of the head of the operator after the change in position; and
determining the commanded motion further based on the articulation.

11. The computer-assisted device of claim 1, wherein the control unit is further configured to determine the commanded motion by:
determining a first displacement of the head, the first displacement associated with the change in position in a left-right direction relative to the reference;
determining, based on the first displacement, a second displacement by which to adjust the FOV of the imaging device in a yaw direction relative to a FOV reference pose; and
determining the commanded motion based on the second displacement.

12. The computer-assisted of claim 1, wherein the threshold amount comprises a threshold angle.

13. (canceled)

14. The computer-assisted device of claim 1, wherein the control unit is further configured to determine the commanded motion by:
determining an adjustment to the FOV of the imaging device that would align a direction of the FOV of the imaging device with a direction of view of the head of the operator after the change in position; and
determining the commanded motion further based on the adjustment to the FOV of the imaging device.

15. The computer-assisted device of claim 1, wherein the control unit is further configured to:
in response to determining that the head motion signals indicate that the change in position exceeds the threshold amount in the direction, command the at least one of the repositionable structure or the imaging device to adjust the FOV of the imaging device in accordance with a commanded movement associated with a maximum adjustment of the FOV of the imaging device in the direction.

16. (canceled)

17. The computer-assisted device of claim 1, wherein the control unit is further configured to:
in response to determining that the head motion signals indicate that the change in position exceeds the threshold amount in the direction for at least a threshold period of time, reset the reference based on a current position of the head.

18-20. (canceled)

21. The computer-assisted device of claim 1, wherein the commanded motion is further determined based on control signals from at least one of a hand-operated or a foot-operated input device.

22. (canceled)

23. A method comprising:
receiving head motion signals indicative of a change in position of a head of an operator relative to a reference, and
in response to determining that the head motion signals indicate that the change in position does not exceed a threshold amount in a direction, causing an orientation of a field of view (FOV) of an imaging device to be adjusted in accordance with a commanded motion by commanding movement of at least one of the imaging device or a repositionable structure supporting the imaging device, wherein the commanded motion is determined based on the change in position.

24. The method of claim 23, further comprising determining the commanded motion by:
determining a first angle associated with the change in position in a left-right direction relative to the reference;
determining, based on the first angle, a second angle by which to adjust the FOV of the imaging device in a yaw direction relative to a FOV reference pose; and
determining the commanded motion based on the second angle.

25-26. (canceled)

27. The method of claim 24, further comprising determining the commanded motion further based on a fourth angle, or a fifth angle, or a second displacement, wherein:
the fourth angle is determined based on a third angle associated with the change in position in an up-down direction relative to the reference;
the fourth angle is used to adjust the FOV of the imaging device in a pitch direction relative to the FOV reference pose;
the fifth angle is determined based on the change in position in the left-right direction relative to the reference, and the fifth angle is used to determine a roll the FOV of the imaging device; or
the second displacement is determined based on a first displacement of the head associated with the change in position in the left-right direction relative to the reference, and the second displacement is used to adjust the FOV of the imaging device in the yaw direction relative to the FOV reference pose.

28-30. (canceled)

31. The method of claim 23, wherein the threshold amount comprises a threshold angle.

32. The method of claim 23, further comprising: determining the commanded motion by:
determining an adjustment to the FOV of the imaging device that would align a direction of the FOV of the imaging device with a direction of view of the head of the operator after the change in position; and
determining the commanded motion further based on the adjustment to the FOV of the imaging device.

33. The method of claim 23, further comprising: in response to determining that the head motion signals indicate that the change in position exceeds the threshold amount in the direction:
commanding at least one of the repositionable structure or the imaging device to adjust the FOV of the imaging device in accordance with a commanded movement associated with a maximum adjustment of the FOV of the imaging device in the direction; or
resetting the reference based on a current position of the head.

34-36. (canceled)

37. One or more non-transitory machine-readable media comprising a plurality of machine-readable instructions which when executed by one or more processors are adapted to cause the one or more processors to perform a method comprising:
receiving head motion signals indicative of a change in position of a head of an operator relative to a reference, and
in response to determining that the head motion signals indicate that the change in position does not exceed a threshold amount in a direction, causing an orientation of a field of view (FOV) of an imaging device to be adjusted in accordance with a commanded motion by commanding movement of at least one of the imaging device or a repositionable structure supporting the imaging device, wherein the commanded motion is determined based on the change in position.

38. The one or more non-transitory machine-readable media of claim 37, wherein the method further comprises: determining the commanded motion by determining the commanded motion further based on a fourth angle, or a fifth angle, or a second displacement, wherein:
the fourth angle is determining based on a third angle associated with the change in position in an up-down direction relative to the reference, and the fourth angle is used to adjust the FOV of the imaging device in a pitch direction relative to a FOV reference pose; or
the fifth angle is determined based on the change in position in a left-right direction relative to the reference, and the fifth angle is used to determine a roll the FOV of the imaging device; or
the second displacement is determined based on a first displacement of the head associated with the change in position in the left-right direction relative to the reference, and the second displacement is used to adjust the FOV of the imaging device in a yaw direction relative to the FOV reference pose.

39. The one or more non-transitory machine-readable media of claim 37, wherein the method further comprises: in response to determining that the head motion signals indicate that the change in position exceeds the threshold amount in the direction:
commanding at least one of the repositionable structure or the imaging device to adjust the FOV of the imaging device in accordance with a commanded movement associated with a maximum adjustment of the FOV of the imaging device in the direction; or
resetting the reference based on a current position of the head.
